(19) United States
(12) Patent Application Publication
(10) Pub. No.: US 2011/0052676 A1
(43) Pub. Date: Mar. 3, 2011

(54) Composition For Delaying Cellular Senescence

(76) Inventor: James Vincent Gruber, Washington, NJ (US)

(21) Appl. No.: 12/871,149

(22) Filed: Aug. 30, 2010

Related U.S. Application Data
(60) Provisional application No. 61/238,729, filed on Sep. 1, 2009.

(51) Int. Cl. A61K 9/127; A61K 38/08; A61K 8/64; A61P 43/00; A61P 17/00; A61P 29/00; A61P 39/06; A61P 39/04; A61P 31/00; A61Q 9/04; A61Q 19/02; A61Q 17/04; C12N 5/071

(52) U.S. Cl. 424/450; 514/21.8; 424/60; 424/70.1; 424/62; 435/375; 977/907

(57) ABSTRACT

Disclosed herein is a composition for delaying cellular senescence comprising from about 0.01 wt % to about 5 wt % of Hexapeptide-11(Phe-Val-Ala-Pro-Phe-Pro), based on the total weight of the composition, and a dermatologically-acceptable carrier for the peptide selected from the group consisting of water, oil, alcohol, silicone, and combinations thereof.

### CROSS REFERENCE TO RELATED APPLICATIONS

This application claims the benefit of U.S. Provisional Patent Application Ser. No. 61/238,729, filed on Sep. 1, 2009, the disclosure of which is incorporated herein by reference in its entirety.

### FIELD OF THE INVENTION

The present invention generally relates to compositions for delaying cellular senescence; and more particularly to cosmetic compositions containing hexapeptide-11 effective for delaying intrinsic or stress-induced cellular senescence in skin cells. The present invention also relates to methods for delaying senescence in skin cells.

### BACKGROUND OF THE INVENTION

Most cells cannot divide indefinitely due to replicative or cellular senescence. Replicative or cellular senescence was first observed and proposed as a model for aging at the cellular level about thirty years ago by Hayflick and Moorhead. They made the profound discovery that cells grown in vitro would tend to grow for only 50-60 population doublings, then they reach a point, called replicative senescence, where they cease to produce new DNA but continue to metabolize and produce ATP. Cells that enter replicative senescence will eventually perish, usually through a series of destructive events collectively known as apoptosis.

Cells can become senescent prematurely as a result of stressful events such as toxin, UV radiation, or other oxidative events. This phenomenon is referred to as Stress-Induced Premature Senescence or SIPS.

Since it is believed that cellular senescence is an essential causative element of aging, efforts have been made to develop methods for delaying cellular senescence. For example, US Patent Application Publication 2002/0123526 discloses the use of retinoic acid for delaying cellular senescence in keratinocytes.

US Patent Application Publication 2009/0075902 discloses methods for delaying cellular senescence by employing Nemo Binding Domain (NBD) protein which acts on Nuclear Factor Kappa Beta (NF-κβ) to inhibit activation of NF-κβ proteins, essentially maintaining this key cellular transcription factor in an inactive state.

Recently laid open World Patent Application WO 2009/046436 discloses topical applications of drug rapamycin for delaying cellular senescence through inhibition of mTOR (Mammalian target for rapamycin) genes and pathways.

Won et al discloses in [Science 2005 and Nat Chem Biol 2006] that a drug labeled CGK733, a commercially available synthetic acetamide analogue, was able to actually reverse the “Senescence Clock” thereby converting replicatively senescent cells, in particular fibroblast cells, back into actively replicating cells. The claim for senescence reversal was withdrawn in a later publication by Won et al. See Won et al. Nat Chem Biol 2008.

However, the prior art materials for delaying cellular senescence, such as NBD protein, are expensive to synthesize. Accordingly, they may not be desirable for topical therapeutic applications.

The use of peptides in topical applications and cosmetics is known. For example, U.S. Pat. No. 6,492,326 discloses that pentapeptides can be use to influence skin appearance by stimulating production of collagen expression. Katayama et al. discloses that the same pentapeptides can be used to improve wound healing. [Katayama et al., Biol Chem 1993] Similarly, US Patent Application Publication 2004/0141939 discloses peptides intended for skin care that are suggested to promote adhesion between skin cells. U.S. Pat. No. 5,554,375 discloses a copper-containing peptide suggested to improve damaged skin and wounds and to stimulate hair growth.

There are references in the literature to the use of hexapeptides for topical applications. For example, US20070202216 to Reinhart et al. discloses the use of a hexapeptide of the structure Serine-Isoleucine-Lysine-Valine-Alanine-Valine to improve the appearance of aging skin. US2008152606 to Reinhart describes an acetylated hexapeptide of the structure Acetyl-Glu-Glu-Met-Glu-Arg-Arg to improve the appearance of aging skin. IE20060154 to Laloeuf discloses a hexapeptide of the structure Gly-Pro-Gln-Gly-Pro-Gln for use to improve the appearance of aging skin.

Likewise, peptides derived from yeast extracts, especially extracts from Saccharomyces cerevisiae, also known as Bakers Yeast, can function topically to improve wound healing and the appearance of skin according to Bentley et al., Arch Surg, vol. 125, 1990, 641. A peptide comprising the amino acid sequence Phe-Val-Ala-Pro-Phe-Pro (INCI name: Hexapeptide-11) was isolated from yeast ferments and was reported to firm aging skin [Lupo et al., Dermatol Therapy, vol. 20, 2007, 343.]. This paper does not disclose the amounts of the peptide used for wound healing purposes.

These patents and papers, however, fail to suggest that any of the peptides listed are capable of delaying replicative cellular senescence. Accordingly, there is still a need for cost effective active agents that have the ability to delay cellular senescence. The present invention provides one answer to that need.

### SUMMARY OF THE INVENTION

In one aspect, the present invention relates to compositions for delaying cellular senescence comprising: from about 0.01 wt % to about 5 wt % of hexapeptide-11, based on the total weight of the composition, and a dermatologically-acceptable carrier for the peptide. The carrier is selected from the group consisting of water, oil, alcohol, silicone, and combinations thereof.

In another aspect, the present invention relates to methods for inhibiting intrinsic cellular senescence and stress-induced premature senescence in cells such as, for example, dermal fibroblasts, epidermal keratinocytes and dermal papillae cells. The method includes contacting the cells with a composition containing from about 0.01 wt % to about 5 wt % of hexapeptide-11, based on the total weight of the composition, and a dermatologically-acceptable carrier for the peptide. The carrier is selected from the group consisting of water, oil, alcohol, silicone and combinations thereof.

### BRIEF DESCRIPTION OF THE FIGURES

FIG. 1 is a graph illustrating delayed senescence in intrinsically-aged dermal fibroblasts measured by SA-β-Gal expression

FIG. 2 is a graph illustrating delayed H₂O₂ stress-induced premature senescence in epidermal keratinocytes measured by ATM expression.

FIG. 3 is a graph illustrating delayed H₂O₂ stress-induced premature senescence in epidermal keratinocytes measured by p53 expression.

FIG. 4 is a graph illustrating delayed UV stress-induced premature senescence in dermal papillae cells measured by SA-β-Gal expression.

FIG. 5 is a graph illustrating influence of hexapeptide on cellular expression of DNA repair enzyme Ogg1.

### DETAILED DESCRIPTION OF THE INVENTION

It has now been surprisingly found that hexapeptide, preferably hexapeptide-11, at a concentration of from about 0.01% to about 5%, with a purity of at least 50%, demonstrates an ability to delay intrinsic or stress-induced premature cellular senescence in skin cells as measured by expression of SA-β-Galactosidase (SA-β-Gal), suppression of ATM or p53 or through increased cellular viability as measured by a cell viability assay such as the MTT assay.

As known to those skilled in the art, delaying senescence can be measured by a number of in vitro assays. In particular, expression of SA-β-Galacotsidase, ATM, ATR, p53, p21, and p16 as well as increases in cellular viability as measured by the MTT assay can all be indicative of delays in cellular senescence.

Cellular senescence can also be noted by changes in the morphology of cells that have entered into senescence. Of most interest is diminished expression of SA-β-Galactosidase, a unique cellular marker known to be expressed by cells in either intrinsic or stress-induced cellular senescence.

The cellular expression of the senescence markers can be measured in multiple ways using in vitro assays, but two very practical methods are by human gene microarrays and by Enzyme-Linked Immunosorbent Assays (ELISA). The first technique employs genomic microchips such as those provided by Affymetrix (Santa Clara, Calif.) to examine whether a particular treatment influences the fibroblast's genetic predisposition to create for proteins and enzymes by increasing or decreasing RNA expression. The second test examines the actual expression of the desired senescence proteins by using fluorescently-labeled antibodies specific for the particular protein of interest.

Through extensive and thorough research, it is found for the first time that hexapeptide, particularly hexapeptide-11 is effective in delaying intrinsic or stress-induced cellular senescence in skin cells, such as fibroblasts and dermal papillae cells. For example, studies indicate that hexapeptide can inhibit certain critical cellular protein expressions such as SA-β-Galatosidase, ATM, or p53 cellular protein expression. Hexapeptide has also been found to be able to enhance expression of certain important cellular markers such as the DNA repair enzyme, Ogg1.

Thus, in one embodiment, the present invention provides a composition containing from about 0.01 wt % to about 5 wt %, preferably from about 0.01 to about 2%, more preferably from about 0.1 to about 1%, of a hexapeptide, based on the total weight of the composition, and a dermatologically-acceptable carrier. Preferably, the hexapeptide is hexapeptide-11 (Phe-Val-Ala-Pro-Phe-Pro) having a purity of at least 50%, preferably at least 75%, more preferably at least 90% or greater. The composition is effective in delaying intrinsic or stress-induced cellular senescence in skin cells, especially fibroblasts, keratinocytes and dermal papillae cells.

Fibroblasts are cells that grow in the dermal layer of the skin that are responsible for expression of new collagen and elastin into the skin. Keratinocytes grow in the epidermis of the skin and are responsible for formation of the stratum corneum and lipids in the skin. Dermal Papillae cells also grow in the dermis of the skin and are the cells responsible for expression of hair fibers. Such cells can be grown in culture dishes under conditions known as in vitro to examine beneficial influences of topical treatments.

The compositions of the present invention are useful for topical application and for regulating signs of skin aging, more especially visible and/or tactile discontinuities in skin texture associated with aging. “Regulating the signs of skin aging” includes prophylactically regulating and/or therapeutically regulating one or more of such signs (similarly, regulating a given sign of skin aging, e.g., lines, wrinkles or pores, includes prophylactically regulating and/or therapeutically regulating that sign). As used herein, prophylactically regulating such signs includes delaying, minimizing and/or preventing signs of skin aging. As used herein, therapeutically regulating such signs includes ameliorating, eg, diminishing, minimizing and/or effacing signs of skin aging.

“Signs of skin aging” include, but are not limited to, all outward visibly and tactilely perceptible manifestations as well as any other macro or micro effects due to skin aging. Such signs may be induced or caused by intrinsic factors or extrinsic factors, e.g., chronological aging and/or environmental damage (e.g., sunlight, UV, smoke, ozone, pollutants, stress, etc.). These signs may result from processes which include, but are not limited to, the development of textural discontinuities such as wrinkles, including both fine superficial wrinkles and coarse deep wrinkles, skin lines, facial frown lines, expression lines, rhytides, dermatoheliosis, photodamage, premature skin aging, crevices, bumps, pits, large pores (e.g., associated with adnexal structures such as sweat gland ducts, sebaceous glands, or hair follicles), “orange peel” skin appearance, dryness, scaliness, flakiness and/or other forms of skin unevenness or roughness; excess skin oil problems such as over production of sebum, oiliness, facial shine, foundation breakthrough; abnormal desquamation (or exfoliation) or abnormal epidermal differentiation (e.g., abnormal skin turnover) such as scaliness, flakiness, keratoses, hyperkeratinization; inadequate skin moisturization (or hydration) such as caused by skin barrier damage, environmental dryness; loss of skin elasticity (loss and/or inactivation of functional skin elastin) such as elastosis, sagging (including puffiness in the eye area and jowls), loss of skin firmness, loss of skin tightness, loss of skin recoil from deformation; non-melanin skin discoloration such as undereye circles, blotching (e.g., uneven red coloration due to, e.g., rosacea), sallowness (pale color), discoloration caused by telangiectasia; melanin-related hyperpigmented (or unevenly pigmented) skin regions; post-inflammatory hyperpigmentation such as that which occurs following an inflammatory event (e.g., an acne lesion, in-grown hair, insect/spider bite or sting, scratch, cut, wound, abrasion, and the like); atrophy such as, but not limited to, that associated with aging, steroid use or use of insect, snake or bacterial toxins such as, for example, Botulinum toxin; other histological or microscopic alterations in skin components such as ground substance (e.g., hyaluronic acid, glycosaminoglycans, etc.), collagen breakdown and structural alterations or abnormalities (e.g., changes in the stratum corneum, dermis, epidermis, the skin vascular system such as telangiectasia); the skin nervous system, tissue responses to insult such as itch or pruritus; and alterations to underlying tissues (e.g., subcutaneous fat, cellulite, muscles, trabeculae, septae, and the like), especially those proximate to the skin.

It is to be understood that the present invention is not to be limited to regulation of the above mentioned “signs of skin aging” which arise due to mechanisms associated with skin aging, but is intended to include regulation of said signs irrespective of the mechanism of origin. As used herein, “regulating skin condition” is intended to include regulation of such signs irrespective of the mechanism of origin.

The present invention is especially useful for therapeutically regulating visible and/or tactile discontinuities in mammalian skin texture, including texture discontinuities associated with skin aging. As used herein, therapeutically regulating such discontinuities includes ameliorating, e.g., diminishing, minimizing and/or effacing visible and/or tactile discontinuities in the texture of mammalian skin, to thereby provide improved skin appearance and/or feel, e.g., a smoother, more even appearance and/or feel. Such visible and/or tactile discontinuities in skin texture include crevices, bumps, pores, fine lines, wrinkles, scales, flakes and/or other forms of textural unevenness or roughness associated with skin aging. For example, the length, depth, and/or other dimension of lines and/or wrinkles are decreased, the apparent diameter of pores decreases, or the apparent height of tissue immediately proximate to pore openings approaches that of the interadnexal skin.

The present invention is also especially useful for prophylactically regulating visible and/or tactile discontinuities in mammalian skin texture, including texture discontinuities associated with skin aging. As used herein, prophylactically regulating such discontinuities includes delaying, minimizing and/or preventing visible and/or tactile discontinuities in the texture of mammalian skin, to thereby provide improved skin appearance and/or feel, e.g., a smoother, more even appearance and/or feel.

The compositions of the present invention, including the essential and optional components thereof, are described in detail hereinafter.

The composition of the invention may also be useful in treating baldness. Bahta A W et al. discloses that dermal papillae cells isolated from individuals who are bald or balding were found to be in an advanced state of premature senescence compared to dermal papillae cells isolated from non-balding individuals. See Bahta A W et al., J. Invest Dermatol 128(2009)1088-1094, the content of which is incorporated herein by reference in its entirety. The authors employ measurements of ATM and SA-β-Galactosidase to demonstrate their findings. Accordingly, the composition of the present invention may be effective in treating baldness by inhibiting cellular senescence.

### Essential Components

### Peptide

An essential component of the present invention is a peptide isolated either through biological means such as fermentation or via more classic methods such as solid state or solution phase synthetic chemistry. More particularly, of importance to the present invention are peptides comprising essentially six amino acids, known collectively as hexapeptides. The amino acids of the hexapeptide can be any of the naturally-occurring amino acids or it may comprise amino acids formed through unnatural synthetic processes.

The peptide of the present invention can be further chemically derivatized by methods known to those skilled in the art including, but not limited to, formation of salts, esters, amides, ethers and the like.

The peptide can also be incorporated into delivery systems that can enhance topical penetration of the peptide into the skin. Such delivery systems are well known to those skilled in the art and include, but are not limited to, liposomes, niasomes, nanosomes and the like.

The preferred hexapeptide according to the invention is a hexapeptide originally isolated from yeast ferments known as Hexapeptide-11 (chemical structure: Phe-Val-Ala-Pro-Phe-Pro) [Lupo et al., Dermatol Therapy 2007]. The structure of Hexapeptide-11 is shown schematically below:

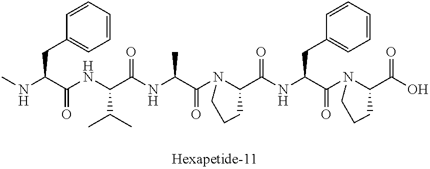

The Hexapeptide-11 of the present invention can also be provided as a synthetic peptide made through standard methods known to those skilled in the art. It has a purity of at least 50%, preferably, 75%, more preferably 90%. Purity of the peptide can be measured in a variety of ways known to those skilled in the art such as NMR, HPLC or GC/MS. Most preferred for the present invention is purity by HPLC.

### Carrier

Another essential ingredient of the present invention is a dermatologically acceptable carrier. The phrase “dermatologically-acceptable carrier”, as used herein, means that the carrier is suitable for topical application to the skin, has good aesthetic properties, is compatible with the actives of the present invention and any other components, and will not cause any untoward safety or toxicity concerns.

The carrier can be in a wide variety of forms. For example, emulsion carriers, including, but not limited to, oil-in-water, water-in-oil, water-in-oil-in-water, and oil-in-water-in-silicone emulsions, are useful herein. These emulsions can cover a broad range of viscosities, e.g., from about 100 cps to about 200,000 cps. These emulsions can also be delivered in the form of sprays using either mechanical pump containers or pressurized aerosol containers using conventional propellants. These carriers can also be delivered in the form of a mousse. Other suitable topical carriers include anhydrous liquid solvents such as oils, alcohols, and silicones (e.g., mineral oil, ethanol, isopropanol, dimethicone, cyclomethicone, and the like); aqueous-based single phase liquid solvents (e.g., hydro-alcoholic solvent systems); and thickened versions of these anhydrous and aqueous-based single phase solvents (e.g., where the viscosity of the solvent has been increased to form a solid or semi-solid by the addition of appropriate gums, resins, waxes, polymers, salts, and the like). Examples of topical carrier systems useful in the present invention are described in the following four references all of which are incorporated herein by reference in their entirety: “Sun Products Formulary” Cosmetics & Toiletries, vol. 105, pp. 122-139 (December 1990); “Sun Products Formulary”, Cosmetics & Toiletries, vol. 102, pp. 117-136 (March 1987); U.S. Pat. No. 4,960,764 to Figueroa et al., issued Oct. 2, 1990; and U.S. Pat. No. 4,254,105 to Fukuda et al., issued Mar. 3, 1981.

The carriers of the present invention can comprise from about 50% to about 99% by weight of the compositions of the present invention, preferably from about 75% to about 99%, and most preferably from about 85% to about 95%.

Preferred cosmetically and/or pharmaceutically acceptable topical carriers include hydro-alcoholic systems and oil-in-water emulsions. When the carrier is a hydro-alcoholic system, the carrier can comprise from about 0% to about 99% of ethanol, isopropanol, or mixtures thereof, and from about 1% to about 99% of water. More preferred is a carrier comprising from about 5% to about 60% of ethanol, isopropanol, or mixtures thereof, and from about 40% to about 95% of water. Especially preferred is a carrier comprising from about 20% to about 50% of ethanol, isopropanol, or mixtures thereof, and from about 50% to about 80% of water. When the carrier is an oil-in-water emulsion, the carrier can include any of the common excipient ingredients for preparing these emulsions. A more detailed discussion of suitable carriers is found in U.S. Pat. No. 5,605,894 to Blank et al., and, U.S. Pat. No. 5,681,852 to Bissett, both of which are herein incorporated by reference in their entirety.

### Optional Components

The compositions of the present invention may optionally comprise additional skin actives. Non-limiting examples of such skin actives include vitamin B3 compounds such as those described in PCT application WO 97/39733, published Oct. 30, 1997, to Oblong et al., herein incorporated by reference in its entirety; hydroxy acids such as salicylic acid; exfoliation or desquamatory agents such as zwitterionic surfactants; sunscreens such as 2-ethylhexyl-p-methoxycinnamate, 4,4′-t-butyl methoxydibenzoyl-methane, octocrylene, phenyl benzimidazole sulfonic acid; sun-blocks such as zinc oxide and titanium dioxide; anti-inflammatory agents; anti-oxidants/radical scavengers such as tocopherol and esters thereof; metal chelators, especially iron chelators; retinoids such as retinol, retinyl palmitate, retinyl acetate, retinyl propionate, and retinal; N-acetyl-L-cysteine and derivatives thereof; hydroxy acids such as glycolic acid; keto acids such as pyruvic acid; benzofuran derivatives; depilatory agents (e.g., sulfhydryl compounds); skin lightening agents (e.g., arbutin, kojic acid, hydroquinone, ascorbic acid and derivatives such as ascorbyl phosphate salts, placental extract, and the like); anti-cellulite agents (e.g., caffeine, theophylline); moisturizing agents; anti-microbial agents; anti-androgens; and skin protectants. Mixtures of any of the above mentioned skin actives may also be used. A more detailed description of these actives is found in U.S. Pat. No. 5,605,894 to Blank et al. (previously incorporated by reference). Preferred skin actives include hydroxy acids such as salicylic acid, sunscreen, antioxidants and mixtures thereof.

Other conventional skin care product additives may also be included in the compositions of the present invention. For example, urea, guanidine, glycerol, petrolatum, mineral oil, sugar esters and polyesters, polyolefins, methyl isostearate, ethyl isostearate, cetyl ricinoleate, isononyl isononanoate, isohexadecane, lanolin, lanolin esters, cholesterol, pyrrolidone carboxylic acid/salt (PCA), trimethyl glycine (betaine), tranexamic acid, amino acids (e.g., serine, alanine, threonine, histidine) and/or their salts, panthenol and its derivatives, collagen, hyaluronic acid, elastin, hydrolysates, primrose oil, jojoba oil, epidermal growth factor, soybean saponins, mucopolysaccharides, and mixtures thereof may be used. Other suitable additives or skin actives are discussed in further detail in PCT application WO 97/39733, published Oct. 30, 1997, to Oblong et al., previously incorporated by reference in its entirety.

### Other Components

The formulation also can comprise other components that may be chosen depending on the carrier, optional components or the intended use of the formulation. Additional components include, but are not limited to antioxidants (such as BHT); emulsion stabilizers (such as carbomer); preservatives (such as phenoxyethanol); fragrances (such as pinene); humectants (such as glycerine); waterproofing agents (such as Fomblins perflouorethers); water-soluble film formers (such as hydroxypropyl methylecellulose); oil-soluble film formers (such as hydrogenated C-9 resins); moisturizing agents (such as cholesterol); cationic polymers (such as Polyquaternium-10); anionic polymers (such as xanthan gum); vitamins (such as tocopherol); and the like.

The compositions can also encompass one or more additional active components, and as such can be either cosmetic or pharmaceutical compositions. Examples of useful actives include, but are not limited to, those that improve or eradicate age spots, keratoses and wrinkles, analgesics, anesthetics, anti-acne agents, antibacterials, antifungals, antiviral agents, antidandruff agents, antidermatitis agents, antipruritic agents, antiemetics, antihyperkeratolytic agents, anti-dry skin agents, antiperspirants, antipsoriatic agents, antiseborrheic agents, anti-aging agents, anti-wrinkle agents, antihistamine agents, sunscreen agents, depigmentating agents, wound-healing agents, anti-inflammatories, tanning agents, or hormones.

Particularly preferred embodiments of the present formulations are skin care lotions or creams used as anti-aging products. To that end, the present formulations are combined with agents that are moisturizers, emollients or humectants. Examples of useful combinations are oils, fats, waxes, esters, fatty acid alcohols, fatty acid ethoxylates, glycols, sugars, hyaluronic acid and hyaluronates, dimethicone, cyclomethicone, and the like. Further examples can be found in the International Cosmetic Ingredient Dictionary CTFA, Tenth Edition, 2004.

The present invention also contemplates the delivery of energy to the skin to enhance the effectiveness of the essential component of the invention, via a delivery enhancement device, to keratinous tissue, either simultaneously and/or sequentially (e.g., within 10 minutes) with application of the topical composition. The energy delivery device may deliver energy in a variety of forms, including but not limited to energy in the form of light, heat, sound (including ultrasonic sound), magnetic energy, electromagnetic energy (including radiofrequency and microwaves), mechanical energy (exfoliating or microdermabrasion device), and combinations thereof.

### Preparation of Compositions

The compositions of the present invention are generally prepared by conventional methods such as are known in the art of making topical compositions. Such methods typically involve mixing of the ingredients in one or more steps to a relatively uniform state, with or without heating, cooling, application of vacuum, and the like.

### Methods for Regulating Skin Condition

Regulating skin condition involves topically applying to the skin a safe and effective amount of a composition of the present invention. The amount of the composition which is applied, the frequency of application and the period of use will vary widely depending upon the level of the peptide and/or other components of a given composition and the level of regulation desired, e.g., in light of the level of skin aging present in the subject and the rate of further skin aging.

In a preferred embodiment, the composition is chronically applied to the skin. By “chronic topical application” is meant continued topical application of the composition over an extended period during the subject's lifetime, preferably for a period of at least about one week, more preferably for a period of at least about one month, even more preferably for at least about three months, even more preferably for at least about six months, and more preferably still for at least about one year. While benefits are obtainable after various maximum periods of use (e.g., five, ten or twenty years), it is preferred that chronic application continue throughout the subject's lifetime. Typically applications would be on the order of about once per day over such extended periods, however application rates can vary from about once per week up to about three times per day or more.

A wide range of quantities of the compositions of the present invention can be employed to provide a skin appearance and/or feel benefit. Quantities of the present compositions which are typically applied per application are, in mg composition/cm² skin, from about 0.1 mg/cm² to about 10 mg/cm². A particularly useful application amount is about 2 mg/cm².

Regulating skin condition is preferably practiced by applying a composition in the form of a skin lotion, cream, gel, emulsion, spray, conditioner, cosmetic, lipstick, foundation, nail polish, or the like which is intended to be left on the skin for some esthetic, prophylactic, therapeutic or other benefit (i.e., a “leave-on” composition). After applying the composition to the skin, it is preferably left on the skin for a period of at least about 15 minutes, more preferably at least about 30 minutes, even more preferably at least about 1 hour, most preferably for at least several hours, e.g., up to about 12 hours.

Any part of the external portion of the face, hair, and/or nails can be treated, e.g., face, lips, under-eye area, eyelids, scalp, neck, torso, arms, hands, legs, fingernails, toenails, scalp hair, eyelashes, eyebrows, etc.

Another approach to ensure a continuous exposure of the skin to at least a minimum level of the peptide of the present invention is to apply the hexapeptide by use of a patch applied, e.g., to the face. Such an approach is particularly useful for problem skin areas needing more intensive treatment. The patch can be occlusive, semi-occlusive or non-occlusive. The peptide composition can be contained within the patch or be applied to the skin prior to application of the patch. The patch can also include additional actives such as chemical initiators for exothermic reactions such as those described in PCT application WO 9701313 to Burkett et al. The patch is preferably left on the skin for a period of at least about 15 minutes, more preferably at least about 30 minutes, even more preferably at least about 1 hour, most preferably at night as a form of night therapy.

Another approach for applying the composition of the present invention is through a rinse-off composition such as, but not limited to, a shampoo, conditioner, body wash, facial scrub, facial peel and the like.

The following examples further describe and demonstrate embodiments within the scope of the present invention. The examples are given solely for the purpose of illustration and are not to be construed as limitations of the present invention, as many variations thereof are possible without departing from the spirit and scope of the invention. All percentages and ratios used herein are by weight of the total composition and all measurements made are at 25 ° C., unless otherwise designated.

### Examples

### Example 1

### Isolation of Hexapeptide through Fermentation from Saccharomyces cerevisiae

Yeast (Saccharomyces cerevisiae) was grown according to the conditions outlined in Jazwinski S M. Methods in Enzymology 182(1990)154-174, which is incorporated in its entirety. Upon completion of the fermentation process, the yeast was isolated by filtration and resuspended in PBS. The microorganisms were ruptured by running the mixture through a microfluidizer to provide a mixture of ruptured yeast cells and cytoplasmic contents. The undissolved components, which included principally cell wall components, were removed by filtration to provide a mixture of water-soluble materials containing peptides, oligopeptides, sugars and polymeric sugars among other components.

The resulting yeast extract was first fractionated for molecular weight distribution using tangential flow filtration employing a membrane filter of nominal molecular weight cut-off at 3000 daltons. The resulting low molecular weight fraction was further fractionated using High Performance Liquid Chromatography using the following conditions: Column: C18 (1.0×250 mm), Mobile Phase: 5% to 80% of a mixture of 0.1% trifluoroacetic acid in water) and 0.0075% trifluoroacetic acid in 70% acetonitrile. Fractions taken from the chromatography column were isolated and the component of the largest fraction was concentrated to provide a fraction containing the hexapeptide-11 (Phe-Val-Ala-Pro-Phe-Pro), the structure being identified via Erdman Degradation to determine the amino acid sequence.

### Example 2

### Isolation of Hexapeptide Through Chemical Synthesis

The hexapeptide described in Example 1 was also synthesized using solid state peptide synthesis techniques well-known to those skilled in the art. The peptide synthesized via solid state synthesis was isolated with a purity of greater than 95% as determined by HPLC chromatography.

### Example 3

### Delayed Senescence in Intrinsically-Aged Dermal Fibroblasts Measured by SA-β-Gal Expression

The peptide isolated from Example 2 was employed to examine the ability of the peptide to delay senescence in dermal fibroblasts aged through a series of population doublings.

### Fibroblast Cell Culture

Human neonatal fibroblasts were obtained after primary culture (passage 1) and seeded into a set of T-75 flasks in 3 ml/flask of Fibroblast Growth Media (FGM) and grown at 37±2° C. and 5±1% CO₂. The cells were expanded through 6 passages (one passage was defined as growing the cells until the flask was confluent and then splitting the cells 1:2, thus one passage was roughly equal to one population doubling). After the 6^{th} passage, the fibroblasts were split into different treatment groups and treated with the various test materials through passage 18. At passage 18 a portion of the fibroblasts were used to assay changes in Senescence Associated-β-Galactosidase (SA-β-Gal), while the remaining fibroblasts were cultured for an additional week (approximately 2 additional passages) in the absence of test materials.

### SA-β-Gal Staining

Prior to staining, the fibroblasts were washed once with PBS and then fixed for approximately 6 minutes in fixing solution (2% formaldehyde and 0.2% glutaraldehyde in PBS). After fixing, the cells were washed three times with PBS, followed by the addition of the staining solution (150 mM NaCl, 2 mM MgCl₂, 40 mM citric acid (pH 6.0), 12 mM NaPO₃, 5 mM potassium ferrocyanide, 5 mM potassium ferricyanide, and 400 μl g/ml X-gal). The cells were then incubated at 37° C. overnight in a non-CO₂ incubator. On the following day the staining solution was removed and replaced with PBS. The cells were then photographed microscopically and the number of stained cells (SA-β-Gal positive) in each field was counted.

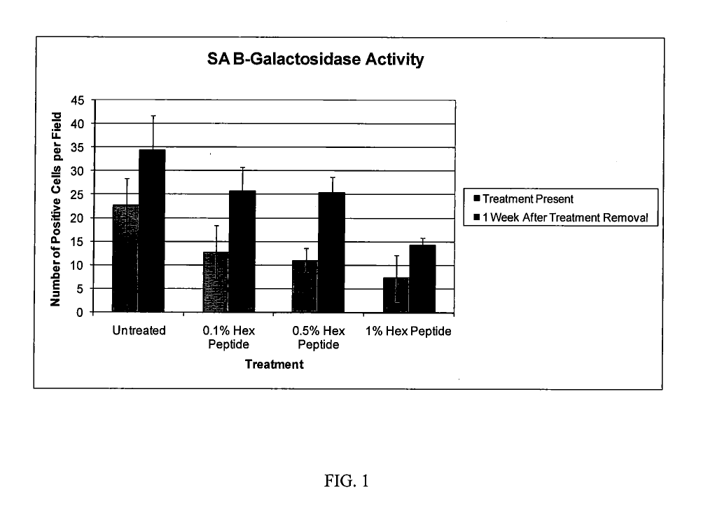

Results of the assay are provided in FIG. 1. These results indicate that after 18 population doublings the expression of SA-β-Gal in the untreated cells was statistically higher than that seen at 0.5 or 1.0% Hexapeptide treatment. This indicates that the hexapeptide was able to delay the onset of senescence in these intrinsically-aged dermal fibroblast cells. One week after removal of the Hexapeptide, the expression of SA-β-Gal returns to normal in all treatments except the 1% treatment where it is shown to increase but not yet back to normal after one week of peptide removal. This data demonstrates that the influence of the peptide on delaying senescence is not permanent and can be reversed upon removal of the peptide from the culture media.

### Example 4

### Delayed H₂O₂ Stress-Induced Premature Senescence in Epidermal Keratinocytes Measured by ATM and p53 Expression

The hexapeptide from Example 2 was used to demonstrate senescence delay in hydrogen peroxide stress-induced prematurely senescent epidermal keratinocytes.

### Human Keratinocyte Cell Culture

Human epidermal keratinocytes were seeded into culture flasks and grown at 37±2° C. and 5±1% CO₂ using serum free Epilife media supplemented as recommended by the manufacturer. When a sufficient number of cells had been grown they were transferred to 96-well plates and cultured for a minimum of 24 hours to allow the cells to adhere to the well plates. After the initial 24-hour incubation, the media was changed to remove any non-adherent cells and the remaining cells were cultured until confluent, with a media change every 48 to 72 hours as needed.

Treatment of Keratinocytes with Test Materials

### H2O2 Treatment

Premature cellular senescence was induced by treating the keratinocytes with H2O2. For the H2O2 treatment, the cell culture media was replaced with phosphate buffered saline (PBS) supplemented with 150 μM of H2O2 . The cells were incubated in the H2O2 solution for two hours, after which they were washed once with PBS and then fresh media, either with or without test material, was applied to the cells. At these levels, the H2O2 treatment was not observed to have an impact on cell viability.

### Analysis of ATM/p53 Expression

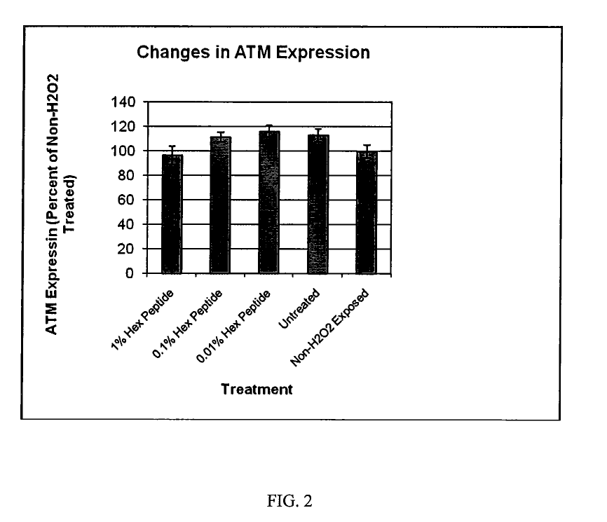

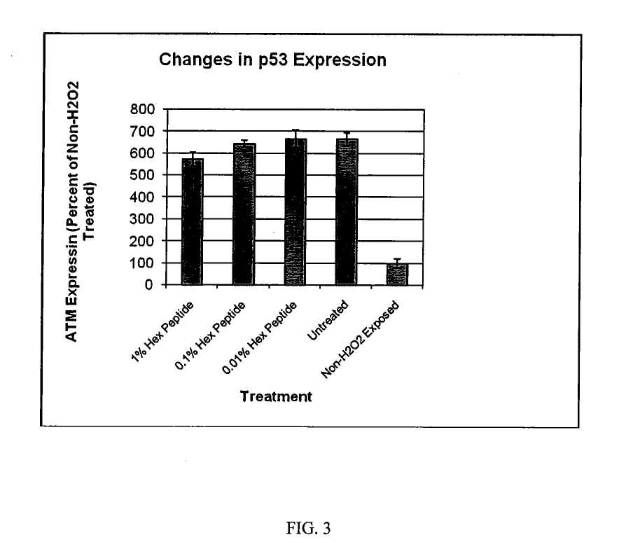

Relative changes in the amount of ATM and p53 expression were determined in the keratinocytes using ELISA based methods. At the end of the treatment period, the cell culture media was removed and replaced with 100 μl/well of ice cold methanol to fix the cells. After fixing, the cells were washed twice with PBS, incubated in 0.5% H2O2 to quench any endogenous peroxidase activity, and then washed two more times in PBS. After washing, 300 μl of blocking solution (1.5% normal goat serum in PBS) was added to each well and the plate was incubated for one hour at room temperature. After blocking, 100 μl of fresh blocking solution containing either anti-ATM or anti-p53 antibody was added, and the well plate was incubated for 1 hour at room temperature. After washing the wells three times with PBS supplemented in 0.05% Tween 20, 100 μl of blocking solution containing an HRP-conjugated anti-goat secondary antibody was added to each well. The plate was incubated for 1 hour at room temperature and then the wells were washed three times with PBS supplemented with 0.05% Tween 20. After the final wash, 200 μl of HRP substrate solution (0.4 mg/ml o-phenylenediamine dihydrochloride, 0.4 mg/ml urea hydrogen peroxide and 0.5 M phosphate-citrate [pH 5.0]) was added to each well and the plate was incubated for 15 to 30 minutes at room temperature. After a sufficient level of color development was achieved the plate was read at 460 nm using a plate reader. Results of the assay are provided in FIGS. 2 and 3.

The data demonstrates that topical application of hexapeptide to prematurely senescent epidermal keratinocytes can statistically delay senescence at the 1% treatment level compared to prematurely senescent untreated keratinocytes as measured by reductions in expression of ATM and p53 proteins.

### Example 5

### Delayed UV Stress-Induced Premature Senescence in Dermal Papillae Cells Measured by SA-β-Gal Expression

The Hexapeptide from Example 2 was used to demonstrate an ability to delay senescence in dermal papillae cells that were induced into premature senescence by treatment with UV radiation.

### Dermal Papillae Cell Culture

Human dermal papillae cells were seeded into 12 well plates in Dermal Papillae Growth Medium (DPGM) and grown at 37±2° C. and 5±1% CO₂ until confluent with a media change every 48 to 72 hours as needed. Once the cells were confluent, the cell culture media was replaced with PBS and the cells were irradiated with 20 mJ/cm² UVB. After the UVB irradiation, the PBS was removed and replaced with cell culture media supplemented with the various test materials. Non-supplemented DPGM was used as the untreated control. One set of cells was not exposed to UVB and served as the non-UVB treated control. After the addition of the media, sets of cells were cultured for 48 hours. At the end of the incubation period the cells were obtained and assayed for changes in SA-β-Gal activity.

In a second set of studies, dermal papillae cells were grown and plated as described above. This second set of cells was treated with the same test materials, only they were not exposed to UVB irradiation. This was done to determine the effects of the test materials alone on the markers measured in this study.

### SA-β-Galactosidase Assay

After the 48 hour incubation, the cells were washed with PBS and then briefly fixed for 6-7 minutes in fixation buffer (2% formaldehyde and 0.2% glutaraldehyde in PBS). The cells were then washed three times with PBS, after which a staining solution was added to the wells (5 mM potassium ferricyanide, 5 mM potassium ferrocyanide, 1 mg/ml X-gal, in phosphate buffer, pH 6.0) and the well plates were incubated overnight at 37° C. (without CO₂). On the following day, the wells were examined microscopically and photographed so that the number of stained cells per field could be quantified. The number of positive staining cells in each field was counted. Mean cell counts for each treatment were then compared using an ANOVA.

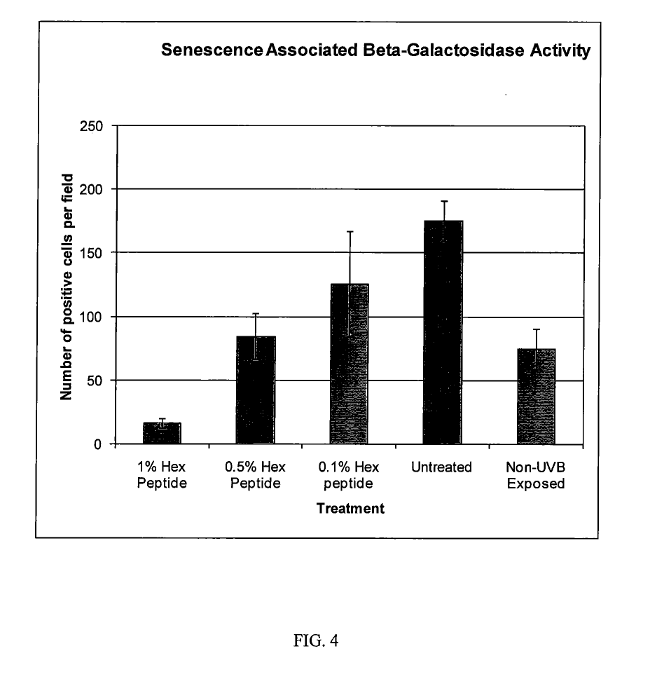

Results of the assay on senescence delay in UV stress-induced prematurely senescent dermal papillae cells are shown in FIG. 4. The results demonstrate that exposure of dermal papillae cells to UV radiation causes an increase in expression of SA-β-Gal indicating the cells are in premature senescence. The application of 0.5 and 1.0% of Hexapeptide demonstrates a statistically significant decrease in SA-β-Gal expression demonstrating delayed senescence in the treated cells.

### Example 6

### Influence of Hexapeptide on Cellular Expression of DNA Repair Enzyme Ogg1

The following example demonstrates the ability of Hexapeptide from Example 2 to upregulate expression of Ogg1, a DNA repair enzyme known to delay senescence in DNA-damaged cells by repairing critical DNA damage before the cells enter into senescence.

### Human Fibroblast Cell Culture

Human fibroblasts were seeded into culture flasks and grown at 37±2° C. and 5±1% CO₂ using FGM. When a sufficient number of cells had been grown they were transferred to 24-well plates and cultured for a minimum of 24 hours to allow the cells to adhere to the well plates. The cells were then grown until confluent, with a media change every 48 to 72 hours.

### Treatment of Fibroblasts

The test materials were prepared in FGM. The media was then removed from the culture plates and replaced with 0.5 ml of media supplemented with test material, with each treatment being tested in triplicate. FGM alone served as the untreated control. After the application of the cell culture media, the plates were incubated for 24 hours at 37±2° C. and 5±1% CO₂. At the end of the incubation period the culture media was removed and the cells were washed once with phosphate buffered saline. After removing the wash, 200 μl of Lysis Buffer (1 mM EDTA, 0.5% Triton X-100, 10 mM NaF, 150 mM NaCL, 20 mM β-glycerophosphate, 1 mM DTT, 10 μg/ml leupeptin, 10 μg/ml pepstatin, 3 μg/ml aprotinin prepared in phosphate buffered saline) was added to the wells and they were incubated on ice for 15 minutes on a rocking platform to lyse the cells. The cell lysates were then transferred to 1.5 ml tubes and centrifuged for 5 minutes at maximum speed (4° C.). The supernatant was retained and stored at −75° C. The protein concentration of the supernatant was determined using the BCA Protein Assay.

### OGG1: Microfiltration Blotting of Cell Lysate and Immunodetection

For each cell lysate sample, 10 μg of protein was combined with 100 μl of Tris Buffered Saline (TBS: 20 mM Tris, pH 7.5, 150 mM NaCl). A PVDF membrane was prewet in methanol, equilibrated with TBS and assembled into the Bio-Dot microfiltration apparatus. After assembly, 100 μl of TBS was added to the wells in the Bio-Dot and the vacuum was applied to ensure that there was an adequate flow through all of the wells. Next, each cell lysate sample prepared above was assigned a well in the apparatus and the sample was applied to the appropriate well. After all of the samples had been added, a vacuum was applied to the apparatus to draw the fluid of the samples through the membrane, leaving the protein adhered to the membrane. TBS was added to wells not assigned a sample to ensure that the membrane did not dry out during the procedure. At the end of the blotting procedure the membrane was removed from the Bio-Dot apparatus, washed in TBS for 5-10 minutes and then placed into blocking solution (TBS with 1% non-fat milk powder) and allowed to incubate for at least 1 hour at room temperature on a rocking platform.

### Antibody Incubation and Detection

After blocking, the membrane was transferred to 20 ml of TBST (TBS with 0.1% Tween-20) and 0.1% non-fat powdered milk with an appropriate dilution of anti-Ogg1 antibody and allowed to incubate overnight at 4° C. on a rocking platform. After this incubation the membrane was washed 3 times (1× for 15 minutes and 2× for 5 minutes) in TBST. The secondary antibody (conjugated with a fluorophore) was then incubated with the membrane in 15 ml of TBST with 0.1% non-fat powdered milk for 1 hour at room temperature and then washed 3 times with TBS (1× 15 minutes, 2× for 5 minutes).

After the final wash, the membrane was placed into a BioRad Molecular Imager FX and scanned using an excitation laser and emission filter combination appropriate for the fluorophore. Images produced by the scanner were then analyzed using ImageJ image analysis software.

### Calculations

### Image Analysis

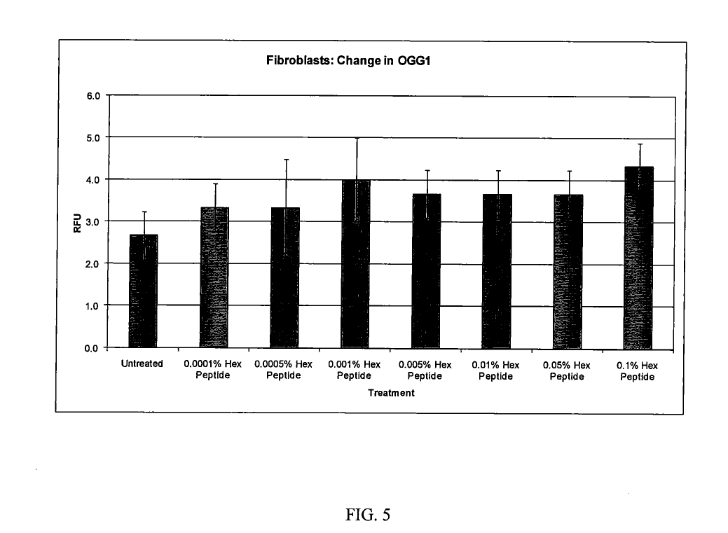

Fluorescence intensity measurements were expressed in Relative Fluorescence Units (RFU). Mean RFU values for each treatment were then calculated and treatments were compared using a one way ANOVA. The results of the Ogg1 assay are shown in FIG. 5.

The results of this assay demonstrate that Hexapeptide can statistically increase expression of Ogg1 at treatment levels of 0.1%. The results from this study demonstrate that Hexapeptide can possibly delay cellular senescence by increasing an important DNA repair enzyme in the cells. Ogg1 is able to replace oxidized guanine residues in damaged DNA, thereby delaying the onset of senescence due to this particular form of DNA damage.

### Example 7

### Oil-in-Water Emulsion with Hexapeptide

The Hexapeptide-11 from Example 2 was formulated into an oil-in-water emulsion using the following formulation and process:

Oil in Water Emulsion With Hexapeptide

| Ingredient | INCI Nomenclature | % |
|---|---|---|
| Phase A | | |
| Water | Water | q.s |
| Versene 100 | Tetrasodium EDTA | 0.10 |
| Phase B | | |
| Glycerin | Glycerin | 2.00 |
| Carbopol Ultrez 10 | Carbomer | 0.20 |
| Phase C | | |
| Brookswax D | Cetearyl Alcohol & Ceteareth-20 | 2.00 |
| Liquiwax DIADD** | Dioctyldodecyl Dodecanedioate | 5.00 |
| Loronate TMP-TC | Trimethylolpropane | 2.00 |
| | Tricaprylate/Tricaprate | |
| Arlacel 60 | Sorbitan Stearate | 1.50 |
| Stearyl Alcohol | Stearyl alcohol | 0.20 |
| Cetyl Alcohol | Cetyl Alcohol | 0.50 |
| Stearic Acid | Stearic Acid | 0.50 |
| Myritol 318 | Caprylic/Capric Triglyceride | 2.00 |
| DC 200/100 cst | Dimethicone | 0.75 |
| Phase D | | |
| Water | Water | 5.00 |
| TEA 99 | Triethanolamine | 0.25 |
| Hexapeptide | Hexapeptide-11 | 1.00 |
| Phase E | | |
| Mikrokill COS | Phenoxyethanol & Caprylyl | 0.75 |
| | Glycol & Chlorphenesin | |

### Procedure:

1. Combine Phase A and heat to 75° C. Mix until uniform.
2. Combine Phase B and heat to 75° C. Mix until uniform.
3. With slow mixing, add Phase B to Phase A. Mix for 20 minutes.
4. Add pre-mix Phase C and mix until uniform. Turn off the heat.
5. In side kettle pre-mix Phase D and add to the batch below 40° C. Mix until uniform.
6. Add Mikrokill COS and fragrance of Phase E, and mix until uniform.

### Example 8

### Water-in-Oil Emulsion Containing Hexapeptide

The hexapeptide from Example 2 was formulated into a water-in-oil emulsion using the following formulation and process:

Water in Oil Emulsion With Hexapeptide

| Ingredient | INCI Nomenclature | % |
|---|---|---|
| Phase A | | |
| Water | Water | q.s to 100 |
| Glycerin | Glycerin | 3.00 |
| Sodium Chloride | Sodium Chloride | 1.00 |
| Hexapeptide | Hexapeptide-11 | 1.00 |
| Phase B | | |
| Mikrokill COS | Phenoxyethanol & Caprylyl | 0.75 |
| | Glycol & Chlorphenesin | |
| SF1328 | Cyclomethicone & Dimethicone | 10.00 |
| | Copolyol | |
| SF 1202 | Cyclomethicone | 8.50 |
| Gel Base Sil | Cyclomethicone & Dimethicone | 1.50 |
| Gel Base BSM-PE | Cyclomethicone & Dimethicone & | 1.50 |
| | Phenyl Trimethicone & | |
| | Polyethylene | |
| | | 100.00 |

### Procedure:

1. Mix all ingredients of Phase A together.
2. Combine Phase B ingredients in order shown, thoroughly mixing each component until homogeneous before adding the next ingredients.
3. Slowly add Phase A to Phase B with good mixing. Gradually increase agitation to high shear as mixture thickens. Continue agitation for 10 minutes.

### Example 9

### Eye Gel Containing Hexapeptide Liposome

The hexapeptide from Example 2 was encapsulated into a liposomal composition, then the encapsulated hexapeptide was incorporated into an eye gel composition using the following formulation and process:

EYE GEL With Hexapeptide liposome

| Ingredient | INCI Nomenclature | % |
|---|---|---|
| Water | Water | Q.S |
| Carbopol Ultrez 21 | Acrylates/C10-30 Alkyl | 0.50 |
| | Acrylate Crosspolymer | |
| Keltrol CG-SFT | Xanthan Gum | 0.10 |
| Butylene Glycol | Butylene Glycol | 5.00 |
| Mikrokill COS | Phenoxyethanol & Caprylyl | 1.00 |
| | Glycol & Chlorphenesin | |
| Dow Corning 193 | Dimethicone Copolyol | 0.30 |
| Surfactant | | |
| Disodium EDTA | Disodium EDTA | 0.10 |
| AMP 95 | Aminomethylpropanol | 0.45 |
| Hexapeptide Liposome | — | 1.00 |

### Procedure:

1. Disperse the Carbopol Ultrez 21 in water at 50° C. and add the Keltrol CG-SFT. Mix until uniform.
2. Add the Butylene Glycol, Mikrokill COS, AMP, EDTA and Silicone 193. Mix until uniform.
3. Add the hexapeptide liposome with sweep agitation at 40° C. Mix until uniform.
5. Adjust pH to 5.5 if necessary.

### Example 10

### Encapsulation of Hexapeptide

The hexapeptide extract from Example 2 was encapsulated into a polymeric matrix using the techniques outlined in US Pat No. 2003/0198682 A1.

### Example 11

### Lipstick Composition

The hexapeptide of Example 2 was formulated into a lipstick using the following formulation and process:

LIPSTICK With Hexapeptide

| Ingredient | INCI Nomenclature | % |
|---|---|---|
| Phase (A) | | |
| Castor Oil | Ricinus Communis | 32.45 |
| | (Castor) Seed Oil | |
| Schercemol TISC | Triisostearyl Citrate | 15.00 |
| Liquiwax PolyIPL | Stearyl PPG-3 Myristyl Ether | 5.00 |
| | Dimer Dilinoleate | |
| Liquiwax PolyEFA | Octyldodecyl PPG-3 Myristyl | 15.00 |
| | Ether Dimer Dilinoloeate | |
| Candelilla Wax | Euphorbia Cerifer | 6.00 |
| | (Candelilla) Wax | |
| Ozokerite 170D | Ozokerite | 2.50 |
| Micro wax SP 19 | Microcrystalline Wax | 3.50 |
| Carnauba Wax | Copernicia cerifera | 1.50 |
| | (carnauba) wax | |
| Methylparaben | Methylparaben | 0.20 |
| Propylparaben | Propylparaben | 0.10 |
| Phase (B) | | |
| Color Grind | | |
| Red 7 Lake c19-7711 | Red 7 Lake | 0.04 |
| Red 6 Lake c19-7712 | Red 6 Lake | 0.17 |
| Red Iron Oxide A-1205 | Iron Oxides | 2.00 |
| Titanium Dioxide Ultra | Titanium Dioxide | 2.00 |
| Fine 70110 | | |
| Black Iron Oxide c33-134 | Iron Oxides | 0.05 |
| Liquiwax PolyEFA* | Octyldodecyl PPG-3 Myristyl | 4.44 |
| | Ether Dimer Dilinoloeate | |
| Phase (C) | | |
| Ascorbyl Palmitate | Ascorbyl Palmitate | 0.05 |
| Flamenco Red | Mica and Titanium Dioxide | 10.00 |
| Hexapeptide | Hexapeptide-11 | 1.00 |

### Procedure:

1. Combine Waxes, Oils and Preservatives (Phase A) and heat to 83°-87° C.
2. Hold temperature and stir until homogeneous.
3. Drop temperature to 75°-80° C., and add Phase B; mix until homogeneous.
4. Add Pearl, hexapeptide and Ascorbyl Palmitate (Phase C).
5. Pour into molds.

### Example 12

### Toner Composition

The hexapeptide of Example 2 was formulated into an aqueous alcoholic tonic using the following formulation and process:

TONER With Hexapeptide

| Ingredient | INCI Nomenclature | % |
|---|---|---|
| Water | Water | Qs. To 100 |
| Betafin BP-20* | Betaine | 3.00 |
| Hexapeptide | Hexapeptide-11 | 1.00 |
| Witch Hazel w/14% | Water & Ethanol & | 25.00 |
| Alcohol | Witch Hazel | |
| Mikrokill COS | Phenoxyethanol & Caprylyl | 0.75 |
| | Glycol & Chlorphenesin | |

### Procedure:

Charge Water and add Betafin BP-20, and hexapeptide. Mix until uniform.
Add Witch Hazel and Mikrokill COS, mix until uniform.

### Example 13

### Body Wash Composition

The hexapeptide of Example 2 was formulated into a body wash using the following formulation and process.

Body Wash With Hexapeptide

| Ingredient | INCI Nomenclature | % |
|---|---|---|
| Water | Water | Q.S |
| Hamp-ene Na2 | Disodium EDTA | 0.10 |
| Glycerin | Glycerin | 2.00 |
| Standapol WAQ-Special | Sodium Lauryl Sulfate | 30.00 |
| Standapol ES-2 | Sodium Laureth Sulfate | 25.00 |
| Cerasynt IP | Glycol Stearate & Stearic | 0.50 |
| | Acid & Aminomethyl Propanol | |
| Velvetex BA-35 | Cocoamidopropyl Betaine | 7.00 |
| Cocamide MEA | Cocamide MEA | 2.00 |
| Mikrokill COS | Phenoxyethanol & Caprylyl | 0.75 |
| | Glycol & Chlorphenesin | |
| Hexapeptide | Hexapeptide-11 | 1.00 |

### Procedure:

1. Heat Water to 70° C. and add Disodium EDTA, Glycerin, and mix until uniform.
2. Keep temperature above 70° C. and add Standapol WAQ Special, Standapol ES-2, Cerasynt IP, Cocamide MEA, Velvetex BA-35, and mix until uniform.
3. Cool to 45° C. and add Mikrokill COS and hexapeptide.
4. Mix until homogenous.

### Example 14

### Fermentation of Hexapeptide

The Hexapeptide from Example 2 was included as part of a fermentation media containing the Yeast Saccharomyces cerevisiae.

A sample of the peptide from Example 2 was placed into an aqueous mixture of Baker's Yeast growth media obtained from Red Star Yeast (Milwaukee, Wis.). The media was inoculated with an active Saccharomyces cerevisiae yeast culture also obtained from Red Star and the mixture was allowed to ferment under controlled aerobic conditions to provide a Live Yeast Cell Derivative (LYCD) obtained using stress conditions as described in U.S. Pat. No. 2,239,345.

### Example 15

### Sub-Micron Emulsion Concentrate

This example illustrates a sub-micron emulsion concentrate that contains hexapeptide prepared as described in Example 2.

| Ingredient | Wt % |
|---|---|
| Trimethylolpropane | 18 |
| Tricaprylate/Tricaprate | |
| Glycerin | 8 |
| Cetearyl Alcohol | 2 |
| Ceteareth 20 | 2 |
| Glyceryl Stearate | 2 |
| BHT | 0.01 |
| Hexapeptide | 1 |
| Water | q.s 100 |

What is claimed is:

1. A composition for delaying cellular senescence comprising from about 0.01 wt % to about 5 wt % of Hexapeptide-11(Phe-Val-Ala-Pro-Phe-Pro), based on the total weight of the composition, and a dermatologically-acceptable carrier for the peptide selected from the group consisting of water, oil, alcohol, silicone, and combinations thereof.

2. The composition of claim 1 wherein the oil is selected from mineral oil, vegetable oil, and combinations thereof.

3. The composition of claim 1, wherein the carrier is an emulsion selected from the group consisting of water-in-oil, oil-in-water, water-in-oil-in-water, and oil-in-water-in-silicone emulsions.

4. The composition of claim 1, wherein the peptide is derived either synthetically or is a component of a fermentation process.

5. The composition of claim 2, wherein the peptide is a synthetic peptide.

6. The composition of claim 1 wherein the hexapeptide is present at a concentration of from about 0.01% to about 2% by weight, based on the total weight of the composition.

7. The composition of claim 1 wherein the hexapeptide is of a purity of at least 50%.

8. The composition of claim 7 wherein the hexapeptide is of a purity of at least 75%.

9. The composition of claim 8 wherein the hexapeptide is of a purity of at least 90%.

10. The composition of claim 1 wherein the hexapeptide is encapsulated in a delivery vehicle selected from the group consisting of liposome, niasome, nanosome, and combinations thereof.

11. The composition of claim 1 further comprising at least one ingredient selected from the group consisting of hydroxyl acids, exfoliation or desquamatory agents, sunscreens, sun-blocks, anti-inflammatory agents, anti-oxidants/radical scanvengers, metal chelators, keto acids, depilatory agents, skin lightening agents, anti-cellulite agents, moisturizing agents, anti-microbial agents; anti-androgens, skin protectants, emulsion stabilizers, preservatives, fragrances, humectants, waterproofing agents, water-soluble film formers, oil-soluble film formers, cationic polymers, vitamins, and combinations thereof.

12. The composition of claim 1, wherein the composition is effective in inhibiting SA-β-Galatosidase, ATM or p53 cellular protein expressions.

13. The composition of claim 1 wherein the composition is effective in enhancing expression of DNA repair enzyme, Ogg1.

14. A method for delaying senescence in skin cells comprising contacting the skin cells with a composition containing from about 0.01 wt % to about 5 wt % of Hexapeptide-11, based on the total weight of the composition, and a dermatologically-acceptable carrier for the peptide selected from the group consisting of water, oil, alcohol, silicone, and combinations thereof.

15. The method of claim 14 wherein the skin cells are fibroblasts, keratinocytes or dermal papillae cells.

16. The method of claim 14 wherein the carrier is an emulsion selected from the group consisting of water-in-oil, oil-in-water, water-in-oil-in-water, and oil-in-water-in-silicone emulsions.

17. The method of claim 14 wherein the hexapeptide is present at a concentration of from 0.01% to about 2% by weight, based on the total weight of the composition.

18. The method of claim 14 wherein the hexapeptide is of a purity of at least 50%.

19. The method of claim 14 wherein the hexpeptide is encapsulated in a delivery vehicle selected from the group consisting of liposome, niasome, nanosome, and combinations thereof.

20. The method of claim 14 wherein the delay of intrinsic or stress-induced cellular senescences in skin cells is measured by expression of SA-β-Galactosidase, suppression of ATM or p53 proteins or through increased cellular viability as measured by a cell viability assay.
